(19) United States
(12) Patent Application Publication
(10) Pub. No.: US 2026/0204400 A1
(43) Pub. Date: Jul. 16, 2026

(54) SYSTEMS AND METHODS FOR NETWORK-BASED PATIENT TRANSFER COORDINATION

(71) Applicant: Salubrity Systems Inc., Minneapolis, MN (US)

(72) Inventors: Benjamin Bryson, Minneapolis, MN (US); Evan Ouimet, Minneapolis, MN (US); Wade Olson, Minneapolis, MN (US)

(73) Assignee: Salubrity Systems Inc., Minneapolis, MN (US)

(21) Appl. No.: 19/446,765

(22) Filed: Jan. 12, 2026

Related U.S. Application Data
(60) Provisional application No. 63/745,057, filed on Jan. 14, 2025.

(51) Int. Cl. G16H 40/20; G06Q 10/02; H04L 9/40

(52) U.S. Cl. CPC G16H 40/20; G06Q 10/028; H04L 63/08

(57) ABSTRACT

An example Network SaaS provides a computer-implemented system and method for coordinating transfers of patients among authenticated healthcare care options, including hospitals, post-acute facilities, outpatient providers, and home-based care environments. The system stores digital care option profiles containing capability attributes and operational capacity indicators, and maintains transfer instances with corresponding transfer-state information. Through a patient transfer portal, a sending user submits patient-associated requirements and a referral packet. A filter module applies configurable criteria to identify candidate destination care options and presents results via a map view and/or list view. A communication module routes messages and referral data through an internal messaging thread and, optionally, encrypted external delivery. A workflow engine updates transfer-state information in response to destination responses and state-transition triggers, while a reservation module generates and resolves reservation conditions. Compliance logging records transfer events with timestamps, and optional analytics and notifications support prioritization and workflow progression.

### CROSS-REFERENCE TO RELATED APPLICATIONS

This application claims the benefit of U.S. Provisional Patent Application No. 63/745,057 , filed Jan. 14, 2025, entitled “Cloud-Based Processing Means for Facilitating Patient Transfers.” The entire disclosure of the provisional application is incorporated herein by reference for all purposes.

### FIELD OF THE INVENTION

This disclosure relates to the field of computer-implemented healthcare information systems, and more particularly to network-based and cloud-enabled software platforms for coordinating patient transfers and care transitions across distributed healthcare care environments.

### BACKGROUND

Transferring patients between healthcare services and facilities such as hospitals, home care environments, outpatient centers, and post-acute care institutions, as well as coordinating initiation of new episodes of care through transfer-related workflows, requires the exchange, evaluation, and routing of large volumes of clinical and operational information across multiple independent systems and organizations. Existing transfer processes generally depend heavily on manual coordination supported by fragmented technology systems. These limitations can make it difficult for healthcare professionals to obtain consistent, real-time operational data required to route patients safely and efficiently. In current practice, discharge and admission workflows often span several disconnected platforms, forcing users to retrieve, verify, and reenter information across siloed systems. The absence of a unified data environment may lead to delays, discrepancies, and inconsistent decision-making, increasing administrative burden and introducing operational risk. These challenges collectively reflect systemic limitations in existing coordination technologies, which lack integrated, machine-readable mechanisms for maintaining shared operational context, validating transfer conditions, and synchronizing decision-making across independent care environments with sufficient speed, traceability, and auditability to support safe and efficient Care Coordination.

One direct consequence of fragmented coordination infrastructure is the inability of care providers to obtain timely, reliable visibility into capacity and resource availability across independent care environments. Waiting times frequently reflect technical constraints. Because current systems often lack reliable mechanisms to share capacity and resource-availability data between facilities, originating providers may have limited or no visibility into when and where capacity exists. Transfer decisions frequently rely on outdated, incomplete, or verbally communicated information that cannot be verified or systematized. This reliance contributes to delayed care progression, increased healthcare occupancy pressures, and repeated follow-up attempts to confirm eligibility or readiness. Limited interoperability and the absence of real-time system-level synchronization across facilities and services may further prevent computer-assisted discovery of available resources or appropriate destinations.

The absence of shared, system-readable capacity information further complicates transfer decisions by forcing originating providers to manually acquire and assess suitability, readiness, and timing across potential destinations. Care options operate under defined capacity limits determined by bed availability, caseload thresholds, staffing resources, equipment readiness, or other operational constraints. However, these constraints are frequently tracked locally using internal systems that do not expose real-time data to external parties. When a transfer request is initiated, staff must manually determine whether a receiving site has the appropriate capabilities, resources, and timing to support that specific transfer. Current systems typically do not enable machine-readable validation of these requirements or structured compatibility assessment, resulting in repetitive communication cycles, manual review, and delays in determining whether a proposed transfer is feasible.

Beyond operational readiness, transfer coordination is further constrained by the lack of structured, interoperable mechanisms for validating payer eligibility, contractual alignment, and network participation across care environments. Insurance and payment workflows introduce additional system limitations. Because transfer eligibility frequently depends on patient-specific coverage requirements, care providers may need to consult multiple payer systems, approval workflows, or paper-based processes to determine whether a transfer is acceptable. These data sources are rarely unified or interoperable, contributing to inconsistent turnaround times and redundant administrative effort. In addition, existing systems provide limited or inconsistent visibility into referral eligibility or contractual alignment between care environments. Originating facilities may lack structured mechanisms to determine whether potential receiving destinations are in-network, contractually partnered, or operationally aligned with the sending organization or payer. As a result, care teams often rely on static or locally maintained lists, undocumented workflow knowledge, or manual verification processes when making referral determinations. Consequently, referral attempts can be routed to destinations that are clinically appropriate but contractually incompatible, operationally incompatible, or financially nonviable, requiring repeated outreach, rework, or redirection. Current technology does not consistently expose these constraints in machine-readable form suitable for consistent evaluative processing across care environments or enable routing logic based on payer network participation, cross-organizational agreements, or facility group membership.

These layered validation gaps place increasing strain on care teams by requiring repeated manual communication and verification across disconnected channels, amplifying workforce and resource constraints. Resource and workforce constraints are exacerbated by limitations in digital coordination systems. In practice, users frequently depend on phone calls, facsimile transmissions, emails, and static data repositories to coordinate transfers across care environments. These channels often cannot preserve transfer context, link communications to system events, or prevent conflicting parallel referrals, requiring transfer status, prior decisions, and current availability to be reverified multiple times and increasing both processing time and opportunities for error.

Taken together, these challenges stem from architectural limitations in existing coordination technologies that prevent shared state awareness, deterministic evaluation, and auditable execution of transfer workflows across independent systems. Current healthcare coordination technologies generally lack a shared, real-time capacity and transfer-state and reservation-condition model capable of coordinating or reconciling multiple concurrent transfer attempts across independent care environments. In the absence of synchronized operational state, parallel transfer requests may be evaluated independently without awareness of competing demand, resulting in conflicting transfer attempts, lost referrals, failed referrals, and repeated manual rework. Because existing systems do not implement evaluation logic tied to shared, real-time capacity state, transfer eligibility cannot be consistently validated or coordinated across simultaneous requests, leading to operational delays and inefficient use of available resources.

These limitations arise in part from the absence of machine-maintained state synchronization and conflict-resolution mechanisms across distributed datasets, preventing concurrent transfer activity from being reconciled in a deterministic manner.

### DEFINITIONS

The following definitions are provided to promote clarity and consistency in describing example embodiments of the disclosed systems and methods. Unless the context clearly indicates otherwise, these definitions are intended to be non-limiting and to encompass variations consistent with the disclosure. The scope of the invention is defined by the claims, which are to be interpreted in light of this specification.

Unless expressly stated otherwise, subsystems, modules, engines, layers, and components described herein may be implemented in software, firmware, hardware, or any combination thereof, and may be deployed in centralized, distributed, virtualized, cloud-hosted, on-premises, or hybrid computing environments. Described embodiments are illustrative and not limiting. Equivalent structures, acts, algorithms, data organizations, or architectural variations that achieve similar results may be used without departing from the scope of the claims.

As used herein, the terms “module,” “engine,” “component,” “layer,” and “subsystem” may refer to logic and/or resources for performing one or more functions and may include one or more processors executing machine-readable instructions, associated memory, data stores, interfaces, and/or specialized hardware, whether co-located or distributed. Such terms do not necessarily imply a discrete physical unit. As used herein, the term “configured to” denotes that a device, system, or component includes structure (e.g., hardware, firmware, and/or executable instructions) that, when operated, performs the recited function.

As used herein, the singular forms “a,” “an,” and “the” include plural referents unless the context clearly indicates otherwise. As used herein, “including,” “includes,” and “include” are open-ended and mean “including, without limitation.” Unless otherwise indicated, terms such as “first,” “second,” and the like are used for convenience and do not imply an order, priority, or relative importance.

As used herein, the term “User” refers to a person, organization, or authenticated system actor authorized to access one or more functions of the Network SaaS in connection with a Care Coordination-related activity. User activities may include initiating, reviewing, modifying, approving, denying, communicating regarding, or otherwise interacting with Transfer-State Information or workflow operations, subject to permissions enforced through the Access Control Module. Users may include individuals acting on behalf of a Care Option, administrative participants, external system actors, or automated system agents authenticated by the platform.

References to sending Users, receiving Users, administrative Users, or other functional User roles describe operational contexts and do not imply distinct User types unless expressly specified.

As used herein, the term “Care Option” refers to an entity, provider, organization, facility, service, operational unit, or resource involved in requesting, delivering, receiving, supporting, or otherwise participating in healthcare services or in a patient transfer or placement workflow. Care Options may include hospitals, post-acute care facilities, outpatient providers, home-based care services, and other care-delivery environments, as well as ancillary or supporting services that participate in or support coordination activities, including transportation, equipment provisioning, administrative services, and similar resources. Care Options are not limited to direct clinical care providers.

As used herein, the term “Care Coordination” refers to a computer-enabled process of organizing, managing, or facilitating healthcare-related services across one or more Care Options or equivalent care environments to support patient needs, transitions, or continuity of care. Care Coordination may include identifying, evaluating, communicating, transferring, matching, reserving, scheduling, routing, exchanging data, validating eligibility, managing operational conditions, and/or managing transfer-state information associated with a patient placement, admission, discharge, referral, or other care workflow. Care Coordination may involve participation by Users, ancillary service providers, insurance or payer entities, or other healthcare-related actors, whether or not such actors are Care Options, and may occur through user interfaces, programmatic interfaces, and/or system integrations. Care Coordination does not require physical movement of a patient or the existence of a traditional care facility, and may include activities that prepare, enable, complete, modify, or cancel a care transfer or related workflow.

As used herein, the term “Transfer Request” refers to a system-generated or User-initiated request to coordinate placement, referral, admission, discharge, or transfer of a patient from an originating Care Option to one or more destination Care Options as part of a Care Coordination process. A Transfer Request may be represented as Structured Data and may result in creation of one or more Transfer Instances, Referral Packets, and/or Transfer-State Records. A Transfer Request may be modified, updated, withdrawn, expired, or otherwise acted upon by the system or by authorized Users.

As used herein, the term “Transfer Instance” refers to a logical system construct representing an individual patient transfer and/or placement coordination workflow, including associated context, participants, data objects, and state progression. A Transfer Instance may maintain associations among patient-related data, Transfer-State Information, reservation data, Messaging Threads, Workflow Events, Operational Indicators, and analytic outputs throughout a lifecycle of the workflow. Multiple Transfer Instances associated with a single patient record or originating Care Option may coexist.

As used herein, the term “Transfer-State Information” refers to machine-maintained data representing current and/or historical status, conditions, and/or progression of a Transfer Instance, including placement states, workflow states, response states, timing states, and operational conditions. Transfer-State Information may be represented using state variables, timestamps, identifiers, flags, enumerated values, metadata, and/or other data structures and may change in response to system logic, workflow sequencing, reservation operations, communications, User actions, and/or analytic outputs.

As used herein, the term “Transfer-State Record” refers to a stored or logically associated representation corresponding to a Transfer Instance that captures Transfer-State Information at one or more points in time. A Transfer-State Record may include current state values and/or historical state values, timestamps, metadata, and identifiers and may be updated, versioned, synchronized, merged, and/or otherwise maintained to support traceability, auditability, workflow execution, and conflict prevention or resolution.

As used herein, the term “State-Transition Trigger” refers to a condition, detected change, or system-recognized signal that causes the Network SaaS to update Transfer-State Information or modify a status of a Transfer Instance in accordance with rule-based sequencing enforced by the Workflow Engine. A State-Transition Trigger may originate from User actions, system logic, reservation updates, analytic outputs, communication activity, timing conditions, external inputs, and/or changes in Operational Capacity Indicators.

As used herein, the term “Workflow Engine” refers to a logic-processing subsystem configured to coordinate and manage sequencing, dependencies, conditions, and/or progression rules associated with Care Coordination workflows and Transfer Instances. The Workflow Engine may evaluate Workflow Events and/or State-Transition Triggers, update Transfer-State Information, and initiate downstream actions including routing instructions, scheduling updates, escalation signals, reservation operations, and/or communication operations. Workflow logic may be automatically or manually advanced, and authorized Users may modify or override workflow progression in accordance with permissions enforced by the Access Control Module.

As used herein, the term “Workflow Event” refers to a system-recognized action, condition, or trigger associated with a Transfer Instance that initiates, advances, modifies, suspends, or completes one or more workflow operations. Workflow Events may originate from User activity, system logic, analytic outputs, reservation updates, communication activity, timing conditions, external inputs, and/or changes in Transfer-State Information.

As used herein, the terms “Structured Dataset” and “Structured Data” refer to data organized in a defined logical format or schema suitable for machine-executed processing, storage, evaluation, exchange, and/or analytics by the system. Structured Data may include normalized fields, mapped attributes, indexed elements, metadata, version identifiers, or other machine-processable representations and may originate from system operations, external integrations, User inputs, and/or automated transformations.

As used herein, the term “Semi-Structured Data” refers to data that does not initially conform to a fixed schema but includes identifiable fields, metadata, tags, or machine-detectable attributes enabling transformation, normalization, and/or mapping into Structured Data by the system. Semi-Structured Data may originate from external information systems, User-provided inputs, and/or automated data sources.

As used herein, the term “Data Repository” refers to a secure storage subsystem configured to maintain Structured Data, Semi-Structured Data, and logically associated information relevant to Care Coordination, including user-provided data and system-generated data. The Data Repository may include one or more relational or non-relational databases, distributed datastores, object stores, and/or other storage mechanisms and may store Digital Care Option Profiles, patient-associated data, Operational Indicators, Transfer-State Information, workflow-related data, communication records, and audit/log data. Stored information may be encrypted, access-controlled, partitioned, and/or otherwise protected in accordance with applicable security requirements.

As used herein, the term “Digital Care Option Profile” refers to a Structured Dataset representing attributes of a Care Option maintained within the Data Repository. A Digital Care Option Profile may include Capability Attributes, Operational Capacity Indicators, staffing details, resource availability, geographic attributes, contact information, operational metrics, historical transfer data, payer compatibility indicators, ancillary service associations, and/or other Structured Data associated with the Care Option. Digital Care Option Profiles may be time-varying, versioned, and/or historically distinguishable.

As used herein, the term “Data Integration Layer” refers to a subsystem configured to exchange Structured Data and/or Semi-Structured Data between the system and external computing systems through programmatic interfaces. The Data Integration Layer may ingest, export, map, normalize, transform, validate, and/or synchronize data used to support Care Coordination workflows, including data associated with Digital Care Option Profiles, Patient-Associated Requirements, Operational Capacity Indicators, Referral Packets, and Transfer-State Information. External computing systems may include electronic health records, health information exchanges, payer systems, ancillary service systems, transportation systems, analytics systems, and other relevant systems.

As used herein, the term “Capability Attribute” refers to a Structured Data element representing a service capacity, clinical accommodation, operational qualification, available resource, ancillary service, or other functional characteristic of a Care Option, as maintained within a Digital Care Option Profile. Capability Attributes may include clinical services, equipment availability, staffing qualifications, specializations, ancillary services, accessibility features, environmental conditions, amenities, and/or other attributes relevant to patient needs or Care Coordination.

As used herein, the term “Operational Indicator” refers to a Structured Data element and/or system-maintained variable representing operational conditions relevant to Care Coordination, including availability, utilization, capability readiness, performance status, workflow progress, and/or other operational state. Operational Indicators may be derived from User inputs, automated calculations, third-party integrations, historical data, and/or system-generated events and may be updated over time.

As used herein, the term “Operational Capacity Indicator” refers to a type of Operational Indicator reflecting resource availability, utilization, and/or readiness conditions relevant to Care Coordination, including availability of beds, staff, equipment, service slots, and/or other capacity elements.

As used herein, the term “Resource Tracking Module” refers to a subsystem configured to detect, monitor, update, and/or synchronize resource availability or utilization associated with Care Options, including staff, equipment, transportation assets, and/or service capacity. The Resource Tracking Module may generate or modify Operational Capacity Indicators using User-entered data, system-derived events, workflow activity, analytic projections, and/or Structured Data received through the Data Integration Layer.

As used herein, the term “Patient-Associated Requirements” refers to Structured Data elements and/or evaluative parameters representing clinical, logistical, administrative, and/or operational conditions associated with a patient for purposes of Care Coordination and transfer coordination. Patient-Associated Requirements may include acuity indicators, service requirements, discharge constraints, payer constraints, timing constraints, and/or other attributes used by the system for machine-executed evaluation.

As used herein, the term “Referral Packet” refers to a Structured Data container comprising patient-associated information, transfer-related metadata, and optional structured, semi-structured, or unstructured artifacts transmitted or exchanged in connection with a Transfer Request or Transfer Instance. A Referral Packet may include clinical details, administrative information, operational context, eligibility information, attachments, and other data required for evaluation or acceptance by a destination Care Option. Referral Packet contents may include regulated patient health information and may be protected using one or more Secure Protocols during storage, transmission, processing, and access.

As used herein, the term “Filter Module” refers to a logic-processing subsystem configured to evaluate and narrow candidate destination Care Options based on one or more Filter Criteria. The Filter Module may be rule-based, algorithmic, statistical, heuristic, machine-learning assisted, or a combination thereof.

As used herein, the term “Filter Criteria” refers to evaluative parameters used to determine suitability and/or prioritization of destination Care Options relative to a Transfer Request or Transfer Instance. Filter Criteria may include Patient-Associated Requirements, Digital Care Option Profile attributes, Operational Capacity Indicators, Transfer-State Information, geographic constraints, Temporal Constraints, system-generated data, analytic outputs, and/or equivalent evaluative inputs and may be weighted, ranked, normalized, and/or otherwise compared.

As used herein, the term “Reservation Module” refers to a subsystem configured to initiate, store, update, synchronize, expire, and/or resolve reservations associated with capacity or availability at a destination Care Option. Reservations may reflect availability of beds, staff, equipment, service slots, transportation resources, ancillary services, or other capacity elements and may incorporate temporal, clinical, operational, and/or resource-related conditions. The Reservation Module may detect and facilitate resolution of conflicting or duplicative reservation conditions through automated logic, User-directed decisioning, or combinations thereof.

As used herein, the terms “Temporal Constraint” and “Availability Window” refer to time-based parameters representing scheduling conditions, resource timing dependencies, discharge or admission timing, prioritization conditions, expiration thresholds, and/or other time-sensitive attributes associated with a Transfer Instance or related workflow. Temporal Constraints and Availability Windows may be static or dynamically updated.

As used herein, the term “Evaluation Result” refers to a machine-generated outcome, score, ranking, suitability assessment, determination, and/or other system-computed result produced through execution of filtering logic, scoring logic, analytics models, rule-based evaluation, and/or equivalent processing. Evaluation Results may be stored, linked to Transfer Instances, displayed through a Visualization Interface, and/or used to guide reservation logic, workflow progression, and/or communications.

As used herein, the term “Analytics Engine” refers to a subsystem configured to perform machine-executed analytic operations and to execute one or more analytic models and/or evaluative logic sets using Structured Data and/or other system-managed data. Analytic operations may include predictive indicators, scoring outputs, statistical evaluations, operational forecasts, prioritization values, trend metrics, and/or other algorithmic results derived from historical and/or real-time data.

As used herein, the term “Scoring Parameters” refers to weighted and/or unweighted evaluative variables used by the Filter Module, Analytics Engine, and/or equivalent logic-processing components to assess Care Option suitability, operational readiness, predicted acceptance likelihood, and/or other transfer-related conditions. Scoring Parameters may represent numerical, categorical, binary, probabilistic, and/or other machine-processable variables and may be aggregated and/or combined to produce comparative evaluations.

As used herein, the term “Communication Module” refers to a subsystem configured to route, transmit, receive, record, and/or otherwise manage Care Coordination-related communications among Users, Care Options, and/or other authorized actors. Communications may include Transfer Requests, Referral Packets, responses, approvals, denials, status updates, attachments, and other information associated with a Transfer Instance. The Communication Module may maintain Messaging Threads linked to Transfer-State Information to support continuity and traceability.

As used herein, the term “Messaging Thread” refers to a logical association between one or more communication events and a corresponding Transfer Instance (or other coordination context) maintained to preserve continuity, context, traceability, and historical integrity of communications. Messaging Threads may include structured or semi-structured communications, attachments, referral information, and/or status indicators and may be time-stamped, versioned, and/or linked to Transfer-State Information.

As used herein, the term “Notification Engine” refers to a subsystem configured to detect events, determine recipients, generate notifications, and deliver or route notifications associated with Care Coordination, workflow progression, operational conditions, system status, and/or User interaction. Notifications may be delivered through in-application alerts, Messaging Thread events, email, SMS, voice, webhooks, and/or other pathways and may be logged or associated with Transfer-State Information.

As used herein, the term “Visualization Interface” refers to an interface subsystem configured to generate and present information to Users in support of Care Coordination activities, including operational data, analytic outputs, workflow status, reservation conditions, Messaging Threads, and Transfer-State Information. The Visualization Interface may include dashboards, lists, tables, charts, map views, calendar views, drill-down displays, and other visual formats and may adapt based on User role, workflow state, and permissions.

As used herein, the term “Patient Transfer Portal” refers to a configurable interaction environment through which authorized Users access and manage transfer coordination operations associated with Transfer Requests and Transfer Instances. The Patient Transfer Portal may provide interfaces to create, review, modify, monitor, and respond to transfer workflows; manage Referral Packets; view Evaluation Results; review Messaging Threads; and perform other workflow actions, subject to authorization rules.

As used herein, the term “Access Control Module” refers to a subsystem configured to authenticate Users, enforce authorization rules, and manage permission structures governing access to system functions and data across one or more Care Options. The Access Control Module may support credential verification, multi-factor authentication, and role-or attribute-based authorization and may enforce multi-tenant isolation.

As used herein, the term “Compliance Logging Module” refers to a subsystem configured to capture, record, store, and maintain logs of system activity, including User actions, authentication events, data access and modification events, reservation updates, transfer-state transitions, Workflow Events, messaging activity, notification events, analytic outputs, and other system state changes, to support auditability, traceability, security monitoring, and compliance. Logged information may include timestamps, identifiers, and relevant metadata and may be protected via encryption, access controls, and/or other security mechanisms.

As used herein, the term “Network SaaS” refers to a network-accessible, computer-implemented coordination environment comprising one or more computing resources, software components, and data repositories configured to facilitate Care Coordination and transfer coordination across multiple Care Options. The Network SaaS may include one or more of the Data Repository, Filter Module, Reservation Module, Communication Module, Notification Engine, Workflow Engine, Analytics Engine, Visualization Interface, Access Control Module, Compliance Logging Module, and Data Integration Layer, and may interoperate with external systems via programmatic interfaces.

As used herein, the term “Network Component” refers to hardware, software, and/or hybrid communication infrastructure configured to enable secure data exchange between Care Options and/or between the system and external computing systems. Network Components may include communication servers, networking resources, APIs, encrypted channels, and equivalent mechanisms providing authenticated connectivity, routing, validation, and transformation of data.

As used herein, the term “Secure Protocol” refers to security controls, transmission standards, and/or data-protection mechanisms configured to protect sensitive, regulated, or otherwise designated data handled by the system. Secure Protocols may include encryption, hashing, signing, tokenization, authenticated channels, role-based authorization, TLS/SSL, PKI, and other approaches. Secure Protocols may be applied variably based on data sensitivity, regulatory classification, operational context, and/or system configuration and may support compliance with applicable regulatory frameworks, including HIPAA where applicable.

### SUMMARY

Disclosed embodiments provide a computer-implemented Network SaaS that coordinates patient transfers and related care-coordination workflows across a plurality of authenticated care options, such as hospitals, post-acute care facilities, outpatient providers, and home-based care environments. In various embodiments, the system includes at least one portal/interface through which authorized users at an originating care option create and manage a transfer instance for a patient and submit patient-associated requirements and a referral packet in structured or semi-structured form.

In one embodiment, the Network SaaS maintains a data repository storing (i) digital care option profiles for participating care options, including capability attributes, operational indicators, and contact endpoints, and (ii) transfer-state information for each transfer instance, including state variables, timestamps, and workflow events. A filter module applies configurable filter criteria to the patient-associated requirements and to the stored care option profiles to generate a candidate set of destination care options, and presents the candidate set through a visualization interface including a map view and/or list view. In certain implementations, the filter criteria includes temporal constraints (e.g., target discharge/admission date) and capability constraints (e.g., required services, equipment, staffing, payer compatibility, or other acceptance conditions), while preserving potentially available destinations when future availability is uncertain.

In one embodiment, the system routes a referral request to one or more selected destination care options using a communication module that generates or updates a messaging thread associated with the transfer instance. The communication module transmits, through an internal messaging channel and/or an encrypted external channel, at least a subset of the referral packet together with metadata identifying the originating care option and a requested transition time window. A workflow engine advances transfer-state information responsive to state-transition triggers including destination responses (accept/deny/request-more-information), timing rules, and user actions, and records associated workflow events. In certain embodiments, a reservation module generates reservation conditions (e.g., bed or resource holds) associated with a transfer instance, detects conflicts between reservation conditions for shared resources, and resolves conflicts via automated rules and/or authorized user decisioning.

In one embodiment, the system produces technical improvements in transfer coordination by maintaining a consistent, machine-executed state model for transfer instances, enforcing access controls and tenant isolation across care options, and generating audit-ready compliance logs that associate transfer communications, state transitions, and reservation actions with user identities and timestamps. In various embodiments, the Network SaaS interoperates with external systems via a data integration layer that ingests and normalizes data from EHR/EMR systems, health information exchanges, or operational systems into structured datasets used by the filter module, workflow engine, and visualization interface. Optional analytics and notification components compute evaluation results (e.g., suitability scores or prioritization outputs) and generate alerts based on workflow state, temporal constraints, and operational indicators to reduce latency in identifying appropriate destinations and to improve traceability of transfer outcomes.

The foregoing summary is provided to introduce representative features and is not intended to limit the scope of the claims.

### BRIEF DESCRIPTION OF THE DRAWINGS

FIG. 1 illustrates a diagrammatic representation of multi-user connectivity between multiple care options and a network-based SaaS providing coordination functionality, in accordance with an embodiment of the present disclosure.

FIG. 2 illustrates a high-level diagrammatic representation of data repositories and coordination features associated with patient-transfer activities and care options, and accessible via a network SaaS, in accordance with an embodiment of the present disclosure.

FIG. 3 illustrates a detailed diagrammatic representation of data and coordination features associated with patients and care options and accessible via a web-based interface of a network SaaS, in accordance with an embodiment of the present disclosure.

FIG. 4 illustrates a screenshot of a web-based interface of a network SaaS presenting a care-options display and associated functionality for identifying and communicating with destination care options, in accordance with an embodiment of the present disclosure.

FIG. 5 illustrates a high-level diagrammatic representation of a system-mediated input-processing-output flow between users and a network SaaS to support care coordination, in accordance with an embodiment of the present disclosure.

FIG. 6 illustrates a detailed flowchart showing a process for transferring patients between care options via a network SaaS, in accordance with an embodiment of the present disclosure.

### DESCRIPTION OF THE INVENTION

In the following discussion, numerous specific details are set forth to provide a thorough understanding of the disclosed subject matter. However, those skilled in the art will appreciate that the present disclosed subject matter may be practiced without such specific details. In other instances, well-known elements, processes or techniques have been briefly mentioned and not elaborated on in order not to obscure the disclosed subject matter in unnecessary detail and description. Moreover, specific details and the like may have been omitted inasmuch as such details are not deemed necessary to obtain a complete understanding of the disclosed subject matter, and are considered to be within the understanding of persons having ordinary skill in the relevant art.

The present disclosure provides computer-implemented systems and methods for coordinating and executing patient-transfer and placement workflows across disparate Care Options using a network-accessible software platform. In one embodiment, the platform maintains, for each transfer request, a system-managed Transfer Instance that stores and updates Transfer-State Information representing a current workflow state, associated timestamps, routing decisions, reservation conditions, messaging context, and other machine-maintained state variables. Each participating Care Option (e.g., a hospital, post-acute facility, outpatient provider, or home-based care service) is onboarded as an authenticated User of the platform via an account creation process in which credentials, permissions, and/or role attributes are established and enforced by an Access Control Module. The system thereby enables originating (sending) Care Options to electronically initiate transfer requests and enables destination (receiving) Care Options to evaluate, accept, deny, request additional information, or otherwise respond through controlled state transitions of the Transfer Instance. As used herein, an originating or sending facility is a Care Option initiating a transfer request for a patient, and a destination or receiving facility is a Care Option selected to receive the patient; however, the system supports iterative or multi-hop transfers in which a patient may be transferred between multiple Care Options over time (e.g., hospital-to-post-acute, post-acute-to-hospital, post-acute-to-home-based care), with each transfer represented as a distinct Transfer Instance or as a linked sequence of Transfer Instances. In contrast to manual, phone-and fax-driven coordination that is prone to inconsistent data, incomplete audit trails, and delayed decisioning, the disclosed platform executes defined workflow logic and secure data exchange to improve traceability, reduce coordination latency, and decrease transfer failures attributable to missing or stale information.

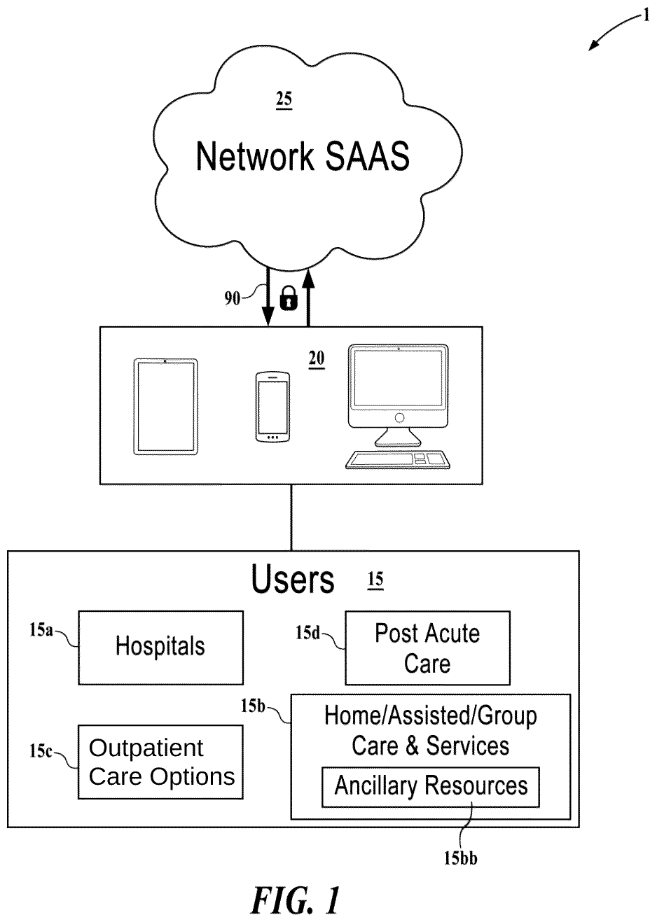

Referring to FIG. 1, the system 1 includes a Network SaaS 25 implemented as one or more computing systems (e.g., cloud-hosted or distributed servers) that provide a centralized coordination environment accessible over a network. In one embodiment, the Network SaaS 25 exposes one or more user interfaces and/or programmatic interfaces that allow authorized Users 15, via respective computing devices 20, to create, view, and update Transfer Instances and associated Referral Packets, and to receive system-generated notifications and workflow prompts. The Network SaaS 25 may further include, as described herein, a Data Repository configured to store Digital Care Option Profiles and transfer-related Structured Data, a Workflow Engine configured to enforce state-transition rules for Transfer Instances, a Communication Module configured to manage messaging and routing of Referral Packets, and a Compliance Logging Module configured to record time-stamped events associated with state changes, data access, and communications for audit reconstruction. By implementing these functions in a network-accessible service model, the system can provide consistent workflow execution and controlled access to transfer-related data while permitting software updates, policy changes, and analytic model updates to be deployed without requiring modification of user devices 20.

In the illustrated embodiment, the Network SaaS 25 securely interconnects a plurality of Care Options 15, including, by way of example, hospitals 15a, home/assisted/group/community living situations 15b, outpatient facilities 15c, and post-acute care facilities 15d, each operating one or more internet-connected computing devices 20. Secure, bidirectional communications (schematically shown as secure communication links 90) may be implemented using Secure Protocols (e.g., authenticated and encrypted channels) such that transfer-related Structured Data, Referral Packets, and workflow updates can be transmitted and received while preserving confidentiality and integrity. Home/assisted/group living situations 15b may include patient homes, senior living communities, supportive living environments, group homes, and similar settings, and the system may further coordinate ancillary resources 15bb that support a transfer or placement workflow, including home care providers, durable medical equipment providers, and medical transportation providers. In one embodiment, each Care Option is represented in the Data Repository by a Digital Care Option Profile comprising Capability Attributes and Operational Indicators, and the system uses such profiles, together with Patient-Associated Requirements associated with a Transfer Instance, to algorithmically narrow candidate destinations and to generate workflow actions (e.g., routing a Referral Packet to a selected subset of Care Options, creating a reservation state, or generating a request-for-information event). The system 1 supports transfer workflows across any combination of origin and destination types (e.g., hospital-to-post-acute, hospital-to-home-based care, post-acute-to-group living, post-acute-to-post-acute), and provides a technical coordination mechanism in which transfer state, communications, and reservation conditions are maintained as machine-executable records rather than as ad hoc human communications, thereby reducing duplicative outreach, improving consistency of decisioning, and enabling deterministic state progression and auditability.

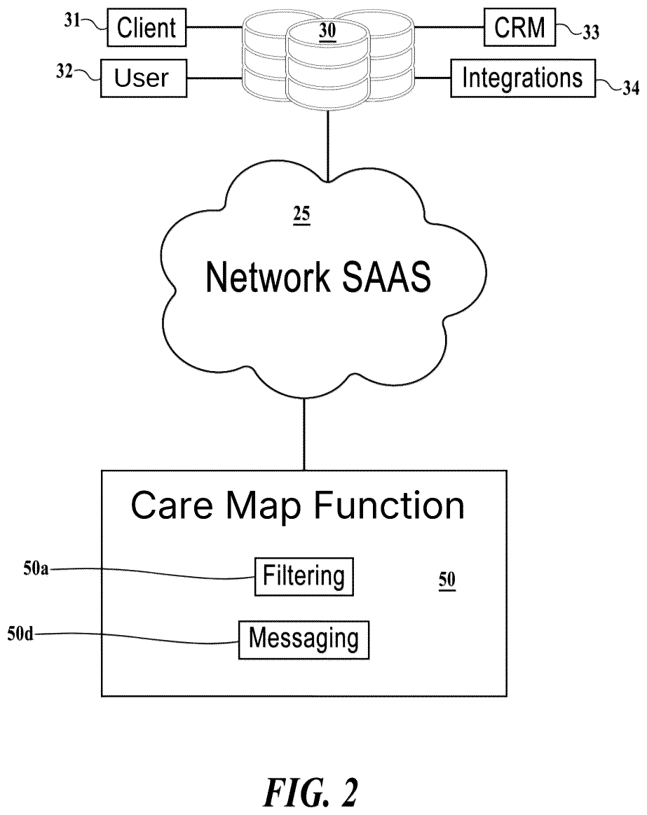

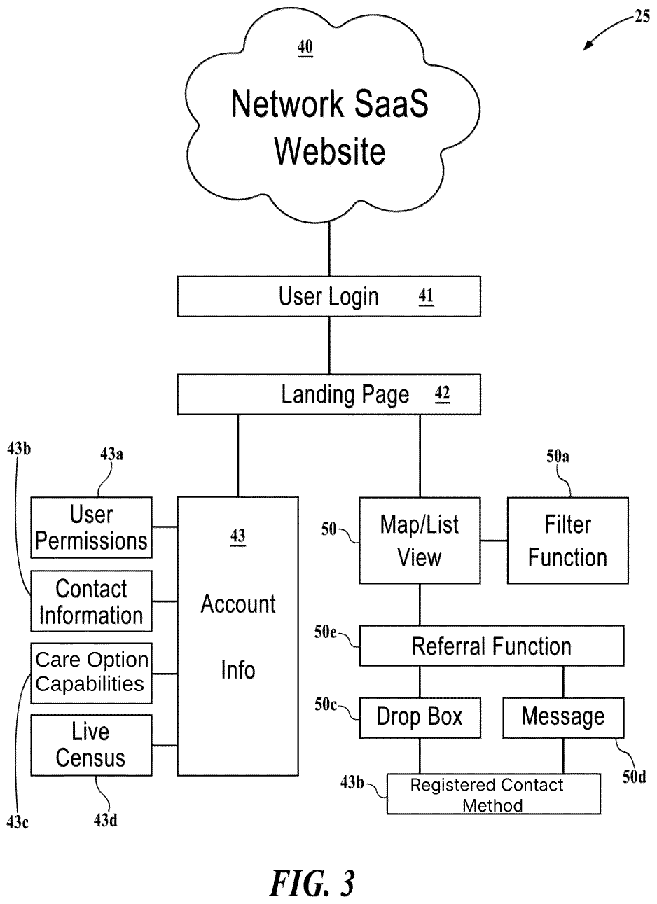

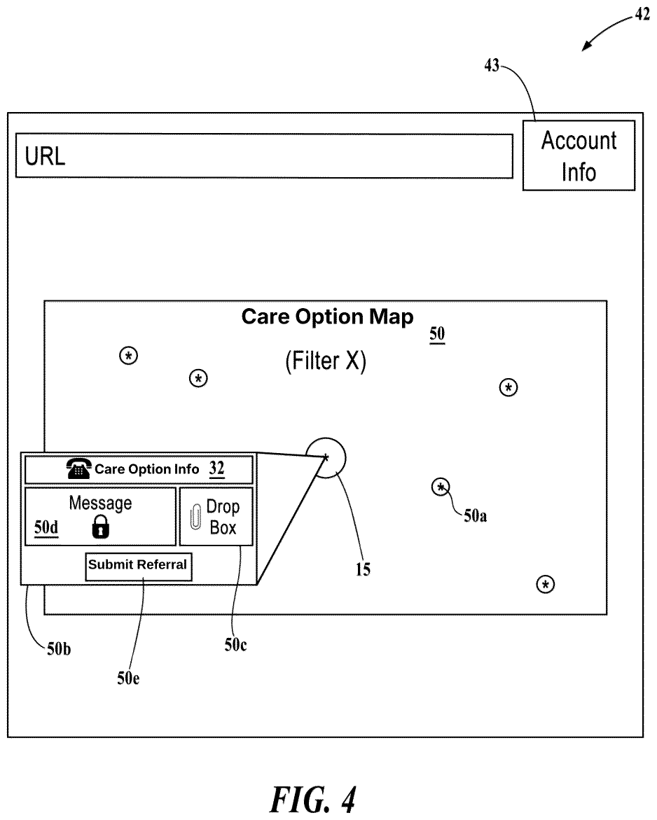

Referring to FIG. 2, the Network SaaS 25 implements a networked coordination environment that interconnects a plurality of Care Options 15 (FIG. 1) and supports execution of patient-transition workflows between such Care Options using system-managed Transfer Instances. In one embodiment, the Network SaaS 25 includes one or more databases 30 (e.g., one or more logical partitions within a Data Repository) that store Structured Data used to operate the platform, including: (i) limited patient/client data 31 sufficient to evaluate placement and coordinate transfers (e.g., non-identifying or minimally identifying attributes, eligibility indicators, acuity level, and/or required services), (ii) facility data 32 (e.g., Digital Care Option Profile attributes, capability indicators, geographic attributes, and contact/routing metadata), (iii) CRM data 33 reflecting relationship and communication context between network participants (e.g., user accounts, messaging history, response times, and interaction metadata), and (iv) patient integration data 34 representing data received via the Data Integration Layer from external systems or exported to external systems in connection with a transfer workflow (e.g., standardized clinical/administrative datasets or mapped fields). The Network SaaS 25 further provides core platform functions that operate on such stored data to facilitate transfer coordination, including a facility-discovery interface 50, a filtering function 50a, and a messaging function 50d. In one embodiment, the facility-discovery interface 50 is dynamically renderable in a map view or a list view and is configured to present candidate Care Options along with capability and availability indicators derived from facility data 32 and/or Operational Indicators maintained by the system. The filtering function 50a applies one or more Filter Criteria to narrow candidate Care Options for a given Transfer Instance based on Patient-Associated Requirements and Care Option Capability Attributes, thereby reducing unnecessary outreach to non-matching destinations. The messaging function 50d provides a controlled communication pathway for exchanging transfer-related messages and Referral Packet information between authenticated Users, and may be implemented as an internal messaging thread within the Network SaaS 25 and/or as an integrated external pathway (e.g., via verified email endpoints associated with registered user accounts, such as registered email 43b of FIG. 3). In the illustrated example, selection of a Care Option within the facility-discovery interface 50 causes the system to render a facility detail panel or pop-up 50b (FIG. 4) that exposes contextual actions, including initiating a message thread via messaging function 50d and applying additional filters via filtering function 50a, thereby enabling a User to evaluate a candidate destination and initiate a controlled, logged workflow interaction without requiring out-of-band communications.

Referring to FIG. 3, a User 15 accesses the Network SaaS 25 through a digital interface such as a website 40 (and/or a native application) that provides authenticated access to transfer-coordination functions. In one embodiment, the website 40 includes a user login function 41 that authenticates the User and establishes a session governed by permissions 43a enforced by an Access Control Module (e.g., role-based and/or attribute-based access). Upon authentication, the User is presented with a landing page 42 that exposes a facility-discovery interface, such as a map/list view function 50. The User may invoke a filtering function 50a to apply Filter Criteria derived from Patient-Associated Requirements for a particular Transfer Instance (e.g., required level of care, capability attributes, payer compatibility, geography, and/or timing constraints), thereby narrowing candidate destination Care Options. When a particular destination is selected, the interface renders a facility detail element (e.g., panel or pop-up) that allows initiation of a referral workflow 50e. In one embodiment, the referral workflow 50e includes a document intake control such as a drop box function 50c for uploading or associating a Referral Packet and a messaging function 50d for establishing a Messaging Thread with the selected destination. The messaging function 50d may be implemented as an internal, system-managed messaging channel within the Network SaaS 25 and/or as an integrated external pathway using verified contact endpoints on file (e.g., registered email 43b and/or other secure routing endpoints), such that communications and attachments can be transmitted under one or more Secure Protocols and logged for auditability. The interface further exposes account information 43 for the User 15, including permissions 43a, contact/routing information 43b, facility capabilities 43c represented as Capability Attributes within a Digital Care Option Profile, and a live census 43d or equivalent operational dataset representing current capacity and utilization conditions. Facility data 32 may include, without limitation, bed availability, staffing and service capability indicators, equipment availability, contact/routing metadata, and other Operational Indicators maintained by the system. Patient/client data 31 is preferably limited to the minimum set of structured attributes needed to evaluate and coordinate placement (and may include de-identified, pseudonymized, or otherwise minimized data fields where feasible), with any regulated information stored, transmitted, and accessed in accordance with configured Secure Protocols and authorization rules.

In addition to transfer initiation and messaging, the Network SaaS 25 may provide registered Care Options with operational and interoperability functions that support transfer coordination and internal facility workflows. In one embodiment, the platform interoperates with external systems such as health information exchanges and electronic medical record systems through a Data Integration Layer configured to ingest, normalize, and map structured or semi-structured datasets into system-defined schemas. The platform may further support administrative and per-user identity management, including multi-user access within a Care Option, permission assignment, and audit logging of access events. The Visualization Interface may render facility operational views such as bed layout views and related utilization analytics based on Operational Capacity Indicators maintained by a Resource Tracking Module. In certain embodiments, an Analytics Engine executes one or more statistical, rule-based, or machine-learning models on platform data (including census, workflow events, and historical transfer outcomes) to generate machine-produced outputs such as projected length-of-stay ranges, projected discharge timing windows, estimated resource utilization, or other operational forecasts; such outputs are stored as analytic results associated with the relevant Transfer Instance and/or Digital Care Option Profile and are presented as decision-support indicators rather than as clinical determinations. The system may also generate automated alerts and notifications (e.g., acceptance/denial status updates, expiring reservation windows, missing referral elements, and/or time-based escalation prompts) via a Notification Engine, and may attribute workflow actions to specific Users with timestamps through a Compliance Logging Module to support traceability, performance measurement, and dispute resolution. In some embodiments, analytic outputs further include actionable operational recommendations (e.g., flagging capacity bottlenecks, routing delays, or mismatches between stated capabilities and observed acceptance outcomes) generated from identified patterns in logged workflow events and operational indicators.

Using the patient transfer system 1, an originating Care Option can generate or update a Transfer Request by assembling Patient-Associated Requirements and referral metadata from one or more sources, including User-entered fields, live census data 43d, and integrated external datasets received through the Data Integration Layer. The system stores such information as structured objects in one or more databases 30 and creates a corresponding Transfer Instance that is advanced through defined states by a Workflow Engine (e.g., pending outreach, awaiting response, reserved, accepted, denied, expired, cancelled, or completed). The originating Care Option can apply filters and sorting operations to candidate destinations based on the Transfer Instance requirements and can render candidates in a map view, list view, calendar view, and/or dashboard view that aggregates operational indicators and workflow status. In certain embodiments, the system also provides CRM-type context that is computationally derived from historical interactions (e.g., prior response times, acceptance likelihood for comparable cases, and communication history) and surfaces such context during destination selection and outreach. The platform may support internal movement workflows (e.g., within a single multi-site organization) and/or “fast-track” pathways in which pre-configured templates, pre-filled referral packets, and/or rule-based routing reduce manual steps for common transfer scenarios. For a destination Care Option, receipt of a Referral Packet and associated message thread causes creation of a corresponding intake record linked to the Transfer Instance, enabling the destination to review requirements, communicate questions, and accept, deny, or reserve capacity under system-managed state transitions. In some implementations, upon creation or update of the intake record, the Analytics Engine generates operational projections (e.g., estimated length-of-stay range or resource impact) based on configured models and available data, and stores such projections with the Transfer Instance for display to authorized Users, thereby providing a concrete, computer-implemented improvement in how multi-facility transfer workflows are coordinated, tracked, and audited across a distributed network.

Referring to FIG. 4, the landing page 42 may present a facility-discovery interface that renders candidate Care Options on a map view 50 (and/or a list view) together with an account information control such as account info tab 43. In the illustrated embodiment, destination facilities 15 within a selected geographic region are initially displayed as selectable indicators (e.g., dots, pins, or other markers), and the set of displayed candidates is reduced or re-ranked after the filtering function 50a is applied. The originating User 15 applies one or more Filter Criteria corresponding to Patient-Associated Requirements for a particular Transfer Instance, which may include a target discharge or transfer date, required capability attributes, payer constraints, distance thresholds, and/or other operational constraints. In certain embodiments, a discharge-date criterion is treated as a soft constraint (i.e., a weighting or eligibility factor) rather than a hard exclusion rule, such that a Care Option may be surfaced as “potentially available” even when the target date is more than a configurable time horizon away, because future capacity is probabilistic and may be represented using predicted or user-updated Operational Capacity Indicators. Selection of a destination Care Option on the map view 50 (or selection of a row in a list view) causes the system to render an interactive facility detail element such as a pop-up box 50b associated with the selected Care Option. In one embodiment, the pop-up box 50b includes (i) facility summary information 32 (including verified contact/routing endpoints such as registered email 43b), (ii) an intake control such as a drop box function 50c for associating or uploading a Referral Packet, (iii) a messaging function 50d that initiates or continues a Messaging Thread, and (iv) a reserve function 50e (e.g., a selectable control) that initiates a capacity reservation request for a specified transition date or availability window.

In one operational example, to request a reservation or initiate a referral, the originating User associates patient transfer materials with the Transfer Instance (e.g., by uploading documents and/or structured fields via the drop box function 50c), provides a destination-facing message via the messaging function 50d, and actuates the reserve function 50e. In response, the Network SaaS 25 generates and transmits a referral request that includes at least: (i) a reference to, or a secure payload comprising, the Referral Packet; (ii) the requested transition date or availability window; and (iii) routing information sufficient to identify the originating Care Option and enable a reply (e.g., an authenticated system messaging endpoint and/or a verified external endpoint). Communications are transmitted using one or more Secure Protocols (e.g., encrypted transport and/or encrypted payloads) and are logged as workflow events associated with the Transfer Instance. Although some implementations may additionally route notifications to an external registered email address, the system preferably maintains a system-of-record Messaging Thread and Transfer-State Record so that acceptance, denial, questions, and subsequent actions can be tracked, time-stamped, and audited without relying on off-platform communications.

Upon receipt of the referral request, the destination Care Option is presented with controls to accept, deny, request additional information, or place a time-bounded reservation, and the Workflow Engine updates Transfer-State Information accordingly. In one embodiment, a denial operation causes the Transfer Instance state to transition to a denied state (optionally with a machine-readable denial reason), and the originating User is notified via the Notification Engine and/or the Messaging Thread. Because a denial does not consume capacity, the system need not modify live census 43d or capacity indicators for the destination based solely on the denial, although the system may log the outcome for analytics (e.g., response-time metrics and acceptance likelihood modeling). The originating Care Option can then continue the facility discovery process using updated filters and/or system-suggested alternatives generated from prior outcomes and operational indicators.

In an acceptance or reservation scenario, the destination Care Option transmits an acceptance message and/or confirms a reservation through the system interface, causing the Transfer Instance to transition to an accepted and/or reserved state. In one embodiment, reservation confirmation creates a reservation record managed by the Reservation Module, optionally including an expiration time and one or more conditions (e.g., receipt of missing documents, payer verification, transport confirmation, or other prerequisites). The originating Care Option may subsequently confirm completion, cancel the request, or modify the transition date (e.g., if the patient is no longer discharging or an alternative placement is selected), and such updates are captured as state transitions and logged workflow events. In certain embodiments, when a reservation becomes binding (e.g., after satisfaction of defined prerequisites or after a confirmation step), the system updates one or more Operational Capacity Indicators for the destination and/or originating Care Option (including live census 43d) to reflect expected inbound and outbound utilization, thereby reducing double-booking and improving coordination across the network.

The account info tab 43 may provide an administrative interface through which an authorized User manages facility configuration, operational indicators, and routing metadata used by the Network SaaS 25. In one embodiment, the account interface includes controls to update live census 43d and/or unit-level capacity indicators, to define capability attributes 43c for one or more units or service lines (e.g., by selecting from structured capability checklists), and to update verified contact endpoints such as registered email addresses and phone numbers used for notifications and routing. The account interface may further display user permissions and roles (e.g., administrator, intake coordinator, clinical reviewer) and provide support contact information or embedded support workflows. Updates entered through the account interface are stored as structured records within the Data Repository, are time-stamped, and may be subject to validation rules and audit logging (e.g., capturing which User made a change, what fields changed, and when), thereby providing a concrete technical mechanism for maintaining accurate operational data that drives filtering, reservation logic, notifications, and workflow sequencing within the Network SaaS 25.

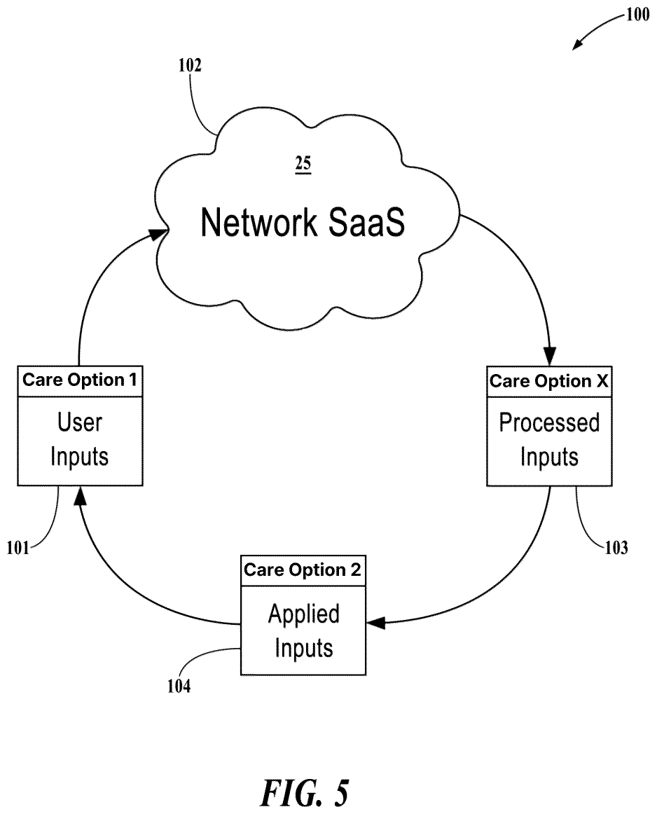

Referring to FIG. 5, a diagrammatic representation illustrates an example method 100 for operating the patient transfer system 1, particularly with respect to facility discovery and execution of the filtering function 50a in connection with a Transfer Instance. In step 101, an originating User 15 associated with an originating Care Option (e.g., “Facility 1”) initiates a search for candidate destination Care Options by providing one or more search parameters via the facility map function 50 and/or a list view. The search parameters may include, by way of example and not limitation, Patient-Associated Requirements (e.g., acuity level, required services, equipment needs, isolation requirements), payer or authorization constraints, geographic constraints, timing constraints (e.g., a desired discharge date or availability window), and/or operational preferences. In step 102, the Network SaaS 25 receives the search parameters through one or more user interface interactions and converts the parameters into a structured query or filter object, optionally applying access-control rules to restrict which destination Care Options are eligible to be returned for the originating User (e.g., based on network membership, contractual relationships, or role-based permissions). In step 103, the Filter Module evaluates the structured query against Structured Data stored in the Data Repository, including Digital Care Option Profiles and current or predicted Operational Capacity Indicators, to generate a candidate set of destination Care Options (e.g., “Facility X”). The Filter Module may apply rule-based eligibility checks and/or scoring logic to rank candidates, including applying hard constraints (must-have requirements) and soft constraints (weighted preferences) that produce Evaluation Results indicating relative suitability. In step 104, the Network SaaS 25 renders one or more resulting destination Care Options (e.g., “Facility 2”) within the map view 50 and/or list view, and enables initiation of a referral or reservation workflow for a selected destination Care Option via the facility detail element 50b. Upon selection, the system can initiate a Messaging Thread and/or transmit a referral request that includes a Referral Packet reference and requested timing information, thereby transitioning the Transfer Instance from a discovery state to a referral-pending, reserved, accepted, or denied state based on subsequent actions by the destination Care Option, as described herein.

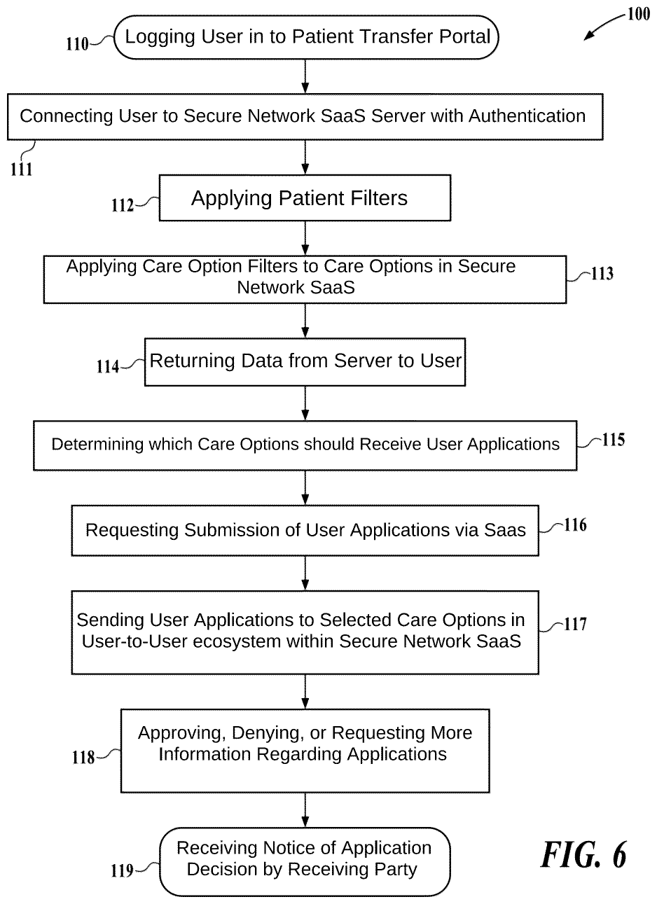

Referring to FIG. 6, a flowchart details an example method 100 for using the patient transfer system 1 via the Network SaaS 25 in a manner that produces and maintains machine-managed Transfer-State Information for a Transfer Instance. In step 110, an originating User 15 associated with an originating Care Option accesses a Patient Transfer Portal (e.g., website 40 and/or a dedicated application interface) and authenticates to the Network SaaS 25. In step 111, the Network SaaS 25 establishes a secure session with the originating User by verifying one or more credentials (e.g., username/password, single sign-on token, multi-factor authentication token, or an API credential) and, responsive to successful authentication, enforces role-or attribute-based permissions that govern which data objects and workflows are accessible to the originating User. In step 112, the originating User creates or updates a Transfer Request for a patient and supplies Patient-Associated Requirements, which may include structured fields representing clinical requirements, logistical constraints, payer or authorization constraints, timing constraints, and/or other transfer-relevant conditions. The Network SaaS 25 stores at least a subset of the Patient-Associated Requirements as Structured Data associated with a Transfer Instance, optionally storing only de-identified or limited patient data sufficient to evaluate placement suitability in a HIPAA-compliant manner. In step 113, the originating User provides destination-selection parameters (e.g., facility attributes, service capabilities, availability indicators, geographic constraints, and discharge or admission timing), and the Network SaaS 25 applies the filtering function 50a by executing the Filter Module against Digital Care Option Profiles, Operational Capacity Indicators, and other facility-associated data maintained in the Data Repository to generate candidate destination Care Options and corresponding Evaluation Results. In step 114, the Network SaaS 25 returns to the originating User a rendered set of candidate destination Care Options (e.g., in a map view or list view), optionally including machine-generated indicators such as suitability scores, availability flags, predicted acceptance likelihood, and/or rationale tags identifying which requirements were satisfied or which constraints were not met. In step 115, the originating User selects one or more destination Care Options for referral and/or reservation, and the Network SaaS 25 creates one or more referral targets within the Transfer Instance and updates Transfer-State Information to reflect that the Transfer Instance is in a referral-selection state or referral-pending state. In step 116, the originating User initiates transmission of one or more referral requests, and the Network SaaS 25 assembles, links, or references a Referral Packet for each selected destination Care Option, the Referral Packet including at least transfer-relevant Structured Data and, in certain embodiments, attachments or external-system references obtained through the Data Integration Layer. In step 117, the Network SaaS 25 transmits, routes, or otherwise makes available the referral request and associated Referral Packet to the selected destination Care Option(s) within the Network SaaS ecosystem, for example by creating a Messaging Thread accessible via an internal messaging interface and/or by generating an encrypted notification to a registered address while maintaining the underlying Referral Packet and Transfer-State Records within the Network SaaS. In step 118, a receiving User associated with a destination Care Option reviews the referral request and generates a response that may include an acceptance, a denial, or a request for additional information, and the Network SaaS 25 records the response as one or more Workflow Events that update Transfer-State Information for the Transfer Instance. In step 119, the Network SaaS 25 generates and delivers a notification to the originating User reflecting the receiving User's response, and stores a time-stamped record of the response and the notification in one or more Transfer-State Records and/or Compliance Logging records such that the transfer workflow can be audited and reconstructed. As described herein, the method 100 is implemented by specific computing components and stored instruction sets of the Network SaaS 25 (e.g., the Data Repository, Filter Module, Communication Module, Notification Engine, Workflow Engine, Access Control Module, and associated interfaces) to provide a concrete, computer-implemented coordination workflow that reduces administrative latency, improves routing accuracy, and maintains consistent transfer-state management across multiple Care Options.

Many variations may be made to the embodiments described herein. All variations are intended to be included within the scope of this disclosure. The description of the embodiments herein can be practiced in many ways. Any terminology used herein should not be construed as restricting the features or aspects of the disclosed subject matter. The scope should instead be construed in accordance with the appended claims.

There may be many other ways to implement the disclosed embodiments. Various functions and elements described herein may be partitioned differently from those shown without departing from the scope of the disclosed embodiments. Various modifications to these implementations may be readily apparent to those skilled in the art, and generic principles defined herein may be applied to other implementations. Thus, many changes and modifications may be made to the disclosed embodiments, by one having ordinary skill in the art, without departing from the scope of the disclosed embodiments. For instance, different numbers of a given element or module may be employed, a different type or types of a given element or module may be employed, a given element or module may be added, or a given element or module may be omitted.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

1. A computer-implemented patient transfer coordination system comprising:
one or more processors and memory implementing a Network SaaS that is network-accessible by a plurality of authenticated Users associated with respective Care Options;
a Data Repository storing (i) Digital Care Option Profiles for the Care Options, the Digital Care Option Profiles including Capability Attributes and one or more Operational Indicators including Operational Capacity Indicators, and (ii) transfer-coordination records including Transfer Instances and corresponding Transfer-State Information;
a Patient Transfer Portal rendered via a Visualization Interface;
an Access Control Module configured to authenticate the Users and enforce authorization rules;
a Filter Module configured to evaluate Filter Criteria based on at least a portion of the Capability Attributes, the Operational Capacity Indicators, and Patient-Associated Requirements associated with a transfer;
a Communication Module configured to create and maintain a Messaging Thread associated with a Transfer Instance and to route transfer-related communications;
a Reservation Module configured to generate, update, and resolve reservation conditions associated with capacity at a destination Care Option;
a Workflow Engine configured to apply rule-based sequencing to update the Transfer-State Information in response to one or more State-Transition Triggers; and
a Compliance Logging Module configured to record workflow events associated with the Transfer Instances; wherein the Network SaaS is configured to:
receive, via the Patient Transfer Portal from a sending User associated with an originating Care Option, a transfer request including Patient-Associated Requirements;
create a Transfer Instance representing the transfer request and store the Transfer Instance in the Data Repository;
determine, using the Filter Module, a set of candidate destination Care Options by applying the Filter Criteria to the Digital Care Option Profiles;
present, via the Visualization Interface, a map view and/or list view identifying the candidate destination Care Options;
receive a selection of a destination Care Option and a referral submission comprising a Referral Packet;
transmit, via the Communication Module, at least a portion of the Referral Packet to the destination Care Option and associate the transmission with the Messaging Thread;
update, via the Workflow Engine, the Transfer-State Information for the Transfer Instance based on a response from the destination Care Option; and
generate, via the Reservation Module and the Workflow Engine, at least one reservation-state update and store a corresponding Transfer-State Record in the Data Repository.

2. The system of claim 1, wherein the Patient Transfer Portal includes a facility information interface that enables a User to edit at least one Operational Capacity Indicator comprising a live census value for a Care Option.

3. The system of claim 1, wherein the map view includes a selectable facility indicator for each candidate destination Care Option and, upon selection, renders a facility pop-up interface that includes (i) destination Care Option contact information, (ii) a document upload control for attaching at least a portion of the Referral Packet, and (iii) a messaging control for composing a message associated with the Messaging Thread.

4. The system of claim 1, wherein the Communication Module is configured to deliver transfer-related communications internally within the Network SaaS and, optionally, to deliver at least a portion of the communications externally via a registered email address stored in the Digital Care Option Profile.

5. The system of claim 1, wherein at least a portion of the transfer-related communications and/or the Referral Packet is stored, transmitted, and accessed using one or more Secure Protocols configured to protect regulated patient-associated information.

6. The system of claim 1, wherein the Filter Criteria include at least one temporal parameter corresponding to an intended discharge date, and wherein the Filter Module is configured to treat the temporal parameter as a non-exclusionary criterion when the intended discharge date is beyond a threshold time window such that candidate destination Care Options remain visible as potentially available.

7. The system of claim 1, wherein the Reservation Module is configured to create a time-bounded reservation condition associated with a destination Care Option and to expire the reservation condition based on a temporal constraint, and wherein expiration generates a State-Transition Trigger processed by the Workflow Engine.

8. The system of claim 1, wherein the Reservation Module is configured to detect conflicting reservation conditions associated with a shared operational resource and to facilitate resolution of the conflicting reservation conditions through automated system logic and/or User-directed decision-making, and to update the Transfer-State Information responsive to the resolution.

9. The system of claim 1, further comprising an Analytics Engine configured to generate an Evaluation Result representing at least one of a suitability score, ranking output, or readiness assessment for candidate destination Care Options, and wherein the Filter Module uses the Evaluation Result to order presentation of the candidate destination Care Options in the map view and/or list view.

10. The system of claim 1, wherein the Compliance Logging Module records, for a given Transfer Instance, at least (i) creation of the Transfer Instance, (ii) transmission of the Referral Packet or a portion thereof, (iii) a reservation-state update, and (iv) an acceptance, denial, or request-for-information response, each with a timestamp.

11. A computer-implemented method of coordinating patient transfers using a Network SaaS, the method comprising:
authenticating, via an Access Control Module, a sending User associated with an originating Care Option; receiving, via a Patient Transfer Portal, Patient-Associated Requirements for a transfer request;
creating, in a Data Repository, a Transfer Instance representing the transfer request and maintaining Transfer-State Information for the Transfer Instance;
applying, via a Filter Module, Filter Criteria to Digital Care Option Profiles comprising Capability Attributes and Operational Capacity Indicators to identify candidate destination Care Options;
presenting, via a Visualization Interface, the candidate destination Care Options in a map view and/or list view;
receiving a selection of a destination Care Option and a referral submission including a Referral Packet;
transmitting, via a Communication Module, at least a portion of the Referral Packet and creating or updating a Messaging Thread associated with the Transfer Instance;
receiving a destination response comprising at least one of accept, deny, or request additional information;
updating, via a Workflow Engine, the Transfer-State Information based on the destination response; and
generating, via a Reservation Module, a reservation-state update associated with capacity at the destination Care Option and storing a corresponding Transfer-State Record.

12. The method of claim 11, wherein presenting the candidate destination Care Options includes rendering a facility pop-up interface that includes a messaging control and a document upload control configured to attach at least a portion of the Referral Packet.

13. The method of claim 11, wherein transmitting includes delivering communications internally within the Network SaaS and, optionally, delivering communications externally to a registered email address stored in a Digital Care Option Profile.

14. The method of claim 11, wherein the Filter Criteria include a discharge-date parameter, and wherein applying the Filter Criteria comprises not excluding a candidate destination Care Option solely based on the discharge-date parameter being more than a threshold time in the future, thereby indicating potential availability.

15. The method of claim 11, further comprising receiving a reservation acceptance through the Patient Transfer Portal and, responsive thereto, generating a State-Transition Trigger that causes the Workflow Engine to transition the Transfer-State Information from a pending state to a reserved state.

16. The method of claim 11, further comprising applying one or more Secure Protocols to encrypt at least a portion of patient-associated information included in the Referral Packet during at least one of storage, transmission, or access.

17. The method of claim 11, further comprising logging, via a Compliance Logging Module, a sequence of workflow events for the Transfer Instance including at least referral submission, destination response, and reservation-state update, with timestamps.

18. The method of claim 11, further comprising updating, via an administrative interface of the Patient Transfer Portal, an Operational Capacity Indicator for at least one Care Option, and thereafter re-applying the Filter Criteria such that the candidate destination Care Options change based at least in part on the updated Operational Capacity Indicator.

19. A non-transitory computer-readable medium storing instructions that, when executed by one or more processors, cause performance of the method of claim 11.

20. The non-transitory computer-readable medium of claim 19, wherein the instructions further cause generation of notifications via a Notification Engine responsive to at least one State-Transition Trigger, the notifications being directed to one or more Users associated with the Transfer Instance.
